(19) United States
(12) Patent Application Publication
(10) Pub. No.: US 2015/0140589 A1
(43) Pub. Date: May 21, 2015

(54) AUTOMATED PROTEIN DIGESTION, RECOVERY, AND ANALYSIS

(71) Applicant: Perfinity Biosciences, Inc., West Lafayette, IN (US)

(72) Inventors: Kevin W. Meyer, West Lafayette, IN (US); Nicholas B. Herold, Lafayette, IN (US); Fred E. Regnier, West Lafayette, IN (US)

(21) Appl. No.: 14/396,272

(22) PCT Filed: Apr. 16, 2013
(86) PCT No.: PCT/US13/36781

§ 371 Date: Oct. 22, 2014

Related U.S. Application Data
(60) Provisional application No. 61/636,705, filed on Apr. 22, 2012.

(51) Int. Cl. C12Q 1/37

(52) U.S. Cl. CPC C12Q 1/37
USPC 435/23; 435/288.6

(57) ABSTRACT

The present disclosure provides an automated system and method for protein digestion, recovery, and analysis. The present disclosure also provides a buffer solution for use therein.

### CROSS REFERENCE TO RELATED APPLICATIONS

This application is a national stage application based on International Patent Application No. PCT/US2013/036781, filed Apr. 16, 2013, which claims priority from U.S. Provisional Patent Application Ser. No. 61/636,705, filed Apr. 22, 2012, the disclosures of which are hereby expressly incorporated by reference herein in their entirety.

### FIELD OF THE DISCLOSURE

The present disclosure relates to protein digestion, recovery, and analysis. More particularly, the present disclosure relates to an automated system and method for protein digestion, recovery, and analysis, and to a buffer solution for use therein.

### BACKGROUND OF THE DISCLOSURE

Understanding the human genome sequence, along with those of a wide variety of animals, plants, insects, and bacteria, has had a major impact on the way proteins are being identified today. Through use of the genetic code and a genomic library, virtually the entire sequence of all proteins in a proteome can be predicted in silico. Moreover, the sequence and molecular weight of peptides derived from proteolytic digests of a protein can be predicted in silico as well using known rules of proteolytic enzyme specificity. Trypsin, for example, is known to be an endopeptidase that cleaves proteins on the C-terminal side of basic amino acid residues. This information has been of huge value in modern proteomics where it is an objective to identify proteins that are undergoing changes in expression and post-translational modification as a function of development, aging, gender, hormonal status, environmental stimuli, or disease.

Tandem mass spectrometry (MS) has become a major tool in this endeavor. This is because i) proteins are easily converted to peptides by enzymes such as trypsin, ii) amino acids and peptide sequence can be identified in terms of their mass based on the above in silico analyses, iii) peptide mixtures are readily ionized in either the electrospray ionization (ESI) or matrix assisted laser desorption ionization (MALDI) modes of transporting peptides into the gas phase and mass analyzed in a 1st dimension of mass discrimination to produce the molecular weight of peptides, iv) after the 1st dimension of mass separation, peptides of a particular mass can be selected and fragmented in the gas phase at peptide bonds to produce fragment ions, v) these fragment ions generally differ by the mass of an amino acid in the peptide sequence, and vi) peptide sequence can be derived from these fragment ions upon mass analysis in a 2nd dimension of MS.

Trypsin is enabling in MS-based proteomics in that it provides multiple peptides that are in general are i) easily analyzed by MS, ii) used to provide a partial or complete sequence of a peptide, iii) traced back to the genome of an organism, iv) utilized in identifying a parent protein that has been expressed by an organism, and v) exploited in many cases to identify post-translational modifications, splice variants, and mutant forms of a protein.

Trypsin digestion is typically carried out in a homogeneous solution of the enzyme with the protein or protein mixture. The ratio of trypsin to protein(s) is kept very low (e.g., 1:100), because otherwise, products from self-digestion of the enzyme are found in the resulting peptide mixture. The reaction is often executed in a solution that denatures the protein(s) so that the locations for cleavage become readily accessible. Typically, the reaction in solution takes place at slightly elevated temperature (e.g. 35° C.) and requires 6-24 hours. In most cases, the proteins are chemically treated prior to digestion to reduce disulfide bridges and to block the resulting thiol groups with alkylation.

Speed and efficacy of the digestion process can be increased by immobilization of the proteolytic enzymes on a solid-support (See, for example, S. Canarelli et al., Hyphenation of multi-dimensional chromatography and mass spectrometry for the at-line-analysis of the integrity of recombinant protein drugs, J. Chromatography B Analyt Technol Biomed Life Sci, 2002, 775(1), pp. 27-35). By immobilization, autodigestion is minimized and the speed of cleavage increases since the enzyme/substrate ratio at the support surface will be much more favorable.

Protein digestion with immobilized trypsin is often carried out with flow-through devices like packed bed reactors or columns sometimes provided as a cartridge. Reaction time is governed by the flow rate and the volume of the device and ranges in practice from 0.25-8 minutes. Alternatively, trypsin is immobilized on a magnetic material or suspended in a spin column format. With these formats reaction time is governed by incubation time.

One issue associated with immobilized trypsin digestion is pH control. The optimal pH for immobilized trypsin digestion is between 7 and 9, such as approximately 8.5. Every pH unit away from 8.5 may result in approximately a 10-fold decrease in activity. As such, buffers may be used to neutralize the digest solution (See, for example, Remco van Soest et al., On-line cHiPLC Based Digestion in nano-LC-MS for Increased Reproducibility, ASMS Poster, Eksigent Technologies—Nanoflex Digestion, 2010; J. R. Freije et al., Chemically modified immobilized trypsin reactor with improved digestion efficiency, J Proteome Res., 2005, 4(5), pp. 1805-13; Y. L. Frank Hsieh et al., Automated Analytical System for the Examination of Protein Primary Structure, Anal. Chem., 1996, 68(3), pp. 455-462). Tris, in particular, has been used in relatively low concentrations of approximately 0.05 M (50 mM) to control pH during trypsin digestion.

Another issue associated with immobilized trypsin digestion is carry-over of the digested materials in the immobilized trypsin or, stated differently, low recovery of the digested materials from the immobilized trypsin (See again van Soest et al., 2010). Prior attempts to reduce carry-over have involved using high concentrations of organic solvents, such as acetonitrile, in digest solution (See again Freije et al., 2005, and Promega Immobilized Trypsin Technical Manual #TM077, 2009). Other prior attempts to reduce carry-over have involved using detergents, such as SDS, and high concentrations of chaotropes, such as guanidine and urea, in the digest solution. However, such digest solutions may negatively impact subsequent desalting and separation steps, such as by inhibiting retention of hydrophilic proteins on hydrophobic columns. Also, such digest solutions may risk damage to downstream MS equipment.

### SUMMARY

The present disclosure provides an automated system and method for protein digestion, recovery, and analysis. The present disclosure also provides a buffer solution for use therein.

According to an exemplary embodiment of the present disclosure, an immobilized enzyme reactor is provided including a protein sample and a digestion buffer solution. The digestion buffer solution includes at least one buffer component having a high buffering capacity, the at least one buffer component present in the digestion buffer solution at a concentration greater than 0.1 M.

According to another exemplary embodiment of the present disclosure, an immobilized enzyme reactor is provided including a protein sample and a digestion buffer solution. The digestion buffer solution includes a solvent and at least one buffer component having a high buffering capacity, the at least one buffer component being present as a majority solute in the solvent.

According to yet another exemplary embodiment of the present disclosure, a method is provided for analyzing a protein. The method includes the steps of preparing a sample from an unfractionated biomatrix comprising the protein, transporting the sample directly to an immobilized enzyme reactor to digest the protein into peptides without first fractionating the unfractionated biomatrix, automatically transporting the peptides from the immobilized enzyme reactor to a desalting apparatus, automatically transporting the peptides from the desalting apparatus to a reverse phase separation apparatus, and analyzing the peptides using a mass spectrometer.

According to still yet another exemplary embodiment of the present disclosure, a system is provided for analyzing a protein. The system includes an immobilized enzyme reactor, a first desalting apparatus in communication with the immobilized enzyme reactor, a second desalting apparatus in communication with the immobilized enzyme reactor and arranged in parallel with the first desalting apparatus, a reverse phase separation apparatus in communication with the first and second desalting apparatuses, and a mass spectrometer in communication with the reverse phase separation apparatus.

### BRIEF DESCRIPTION OF THE DRAWINGS

The above-mentioned and other features and advantages of this disclosure, and the manner of attaining them, will become more apparent and the invention itself will be better understood by reference to the following description of embodiments of the invention taken in conjunction with the accompanying drawings, wherein:

FIG. 1 is a schematic view of an exemplary system for protein digestion, recovery, and analysis;

FIG. 2A is a schematic view of a valve for use in the system of FIG. 1, the valve shown in a first operative position;

FIG. 2B is a schematic view similar to FIG. 2A showing the valve in a second operative position;

FIG. 3 shows a collection of user inputs for operating the system of FIG. 1;

FIG. 4 shows a time program for operating the system of FIG. 1;

FIGS. 5-6C show experimental UV traces for evaluating various digestion buffer solutions; and

FIGS. 7A-7B show experimental SRM results for an insulin assay at various concentrations.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

The present disclosure provides various techniques for improved protein digestion, recovery, and analysis.

### I. Automated System

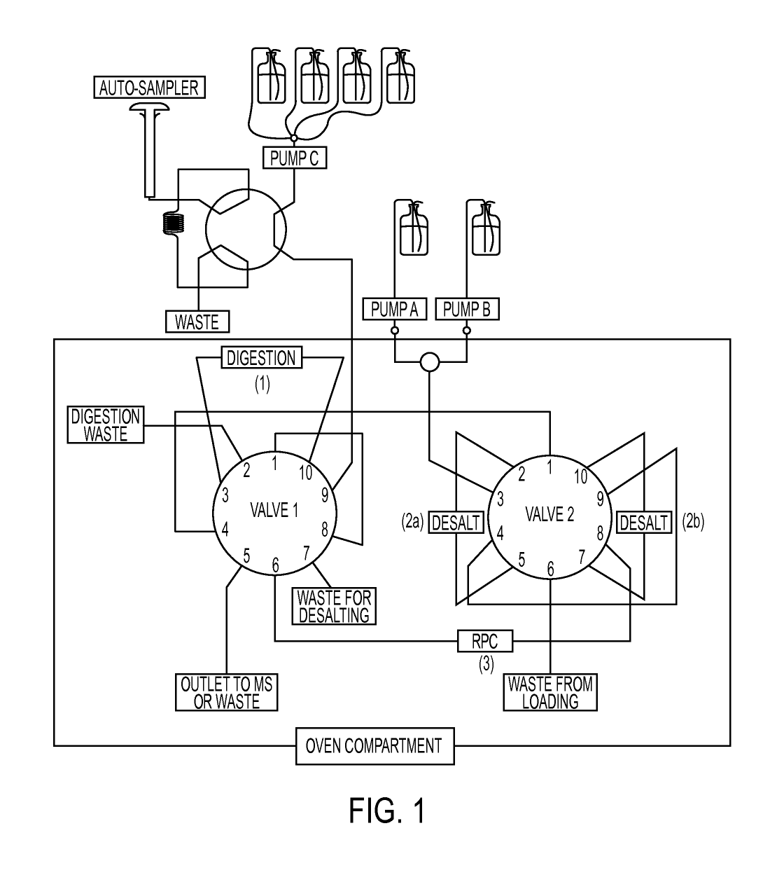

With reference to FIG. 1, an automated system is provided for protein digestion, recovery, and analysis. The system includes an auto-sampler to initiate sample preparation. The system also includes at least three columns coupled in series to the auto-sampler for further processing of the sample: (1) at least one digestion column, (2) at least one desalting column, illustratively two desalting columns (2a) and (2b), and (3) at least one reverse phase chromatography (RPC) column. The columns may be maintained at an elevated temperature (e.g. 35° C.) in an oven compartment. The system further includes a mass spectrometer (MS) coupled in series to the above-described columns via an outlet.

The auto-sampler may hold and house a plurality of sample vials. The auto-sampler may be refrigerated to minimize microbial growth in the sample vials. In addition to the plurality of sample vials, the auto-sampler may also hold and house any necessary solvents or reagents for reduction, alkylation, derivatization, proteolysis, internal standards addition, and dilution, for example, any of which can be aliquoted into sample vials in any order. A pump (Pump C) may be provided to perform solvent selection, which may be a quaternary pump equipped with a solenoid valve.

The system may be initiated by operating a robotic syringe to withdraw an aliquot of one or more desired solutions from the solution vials and to dispense the aliquot into a sample vial. Multiple solutions may be added sequentially to the sample vial, as might be needed in reduction, alkylation, and proteolysis of a sample before analysis. An exemplary solution for addition to the sample vial is a digestion buffer solution, which is described further in Section II below.

After dispensing the solution into the sample vial, the syringe may be cleaned by taking in a suitable rinse solution and dispensing the rinse solution to waste. An exemplary rinse solution is 25 vol. % isopropyl alcohol in water, for example.

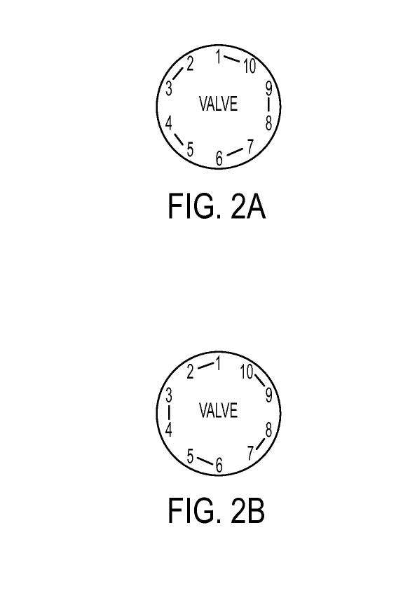

After a suitable incubation time in the sample vial, if any, the syringe withdraws an aliquot of the sample from the sample vial and loads the aliquot onto a first valve (Valve 1), which is illustratively a 10-port valve having a first operative position (FIG. 2A) and a second operative position (FIG. 2B), where solid lines between ports indicate open flow paths. Valve 1 then directs the sample to the downstream digestion column (1). With Valve 1 in the second position (FIG. 2B), the sample illustratively enters Port 9, crosses over to Port 10, and then enters the digestion column (1).

In the digestion column (1), protein in the sample undergoes enzymatic digestion, which breaks the protein apart into more easily identifiable peptide fragments. An exemplary digestion column (1) is an immobilized enzyme reactor (IMER), which includes a proteolytic enzyme immobilized on a suitable solid-support material. The sample may be present in the digestion column (1) for about 1-10 minutes, 1-8 minutes, or 1-5 minutes. The digestion column (1) may be about 30 mm in length, for example.

An exemplary immobilized proteolytic enzyme for use in the digestion column (1) is trypsin. While the most popular proteolytic enzyme is trypsin, other enzymes with alternative functionalities may also be employed, such as Arg-C and Lys-C, so that a variety of peptide products can be generated increasing the protein sequences that are observed and sequenced to provide more definitive identifications.

An exemplary solid-support material for use in the digestion column (1) is a polystyrene/divinylbenzene (PS/DVB) material. Other suitable solid-support materials may include other polystyrene-based materials, silica-based materials, and nitrocellulose-based materials, as well as materials containing a paramagnetic core for sample handling in robotic devices. The solid-support material may be in a particle form, or the solid-support material may be monolithic in form, a membrane, or planar in form. Also, microfluidic channels and the like may be advantageous.

The digested sample from the digestion column (1) returns to Valve 1. With Valve 1 in the second position (FIG. 2B), the digested sample from the digestion column (1) enters Port 3, crosses over to Port 4, and then continues to Port 1 of a second valve (Valve 2). Alternatively, with Valve 1 in the first position (FIG. 2A), the material from the digestion column (1) would enter Port 3, cross over to Port 2, and then continue to waste, such as to rinse the digestion column (1). Like Valve 1, Valve 2 is also illustratively a 10-port valve having a first operative position (FIG. 2A) and a second operative position (FIG. 2B). Valve 2 is illustratively coupled to two desalting columns (2a) and (2b).

In each desalting column (2), the peptide fragments from the digestion column (1) adhere to a hydrophobic surface. Exemplary hydrophobic surfaces are polystyrene divinyl benzene (PS-DVB) and octadecyl silane (C18). Salt ions that are present along with the peptide fragments do not adhere to the desalting column (2) and are washed away to waste. For example, with Valve 2 in the first position (FIG. 2A), the digested sample from the digestion column (1) illustratively enters Port 1, crosses over to Port 10, and then enters the desalting column (2b). Salt ions that do not adhere to the desalting column (2b) illustratively return to Valve 2 via Port 7, cross over to Port 6, and are diverted to waste.

After desalting, the adhered peptide fragments are washed away from the desalting column (2) using a suitable Solvent A that is delivered from Pump A and/or Solvent B that is delivered from Pump B. The solvent make-up is described further below. With Valve 2 now turned to the second position (FIG. 2B), the solvent illustratively enters Port 3, crosses over to Port 4, travels to Port 9, crosses over to Port 10, and then enters the desalting column (2b). Together, the once-adhered peptide fragments and the solvent from the desalting column (2) enter Port 7, cross over to Port 8, and then continue to the RPC column (3).

In the RPC column (3), the peptide fragments are gradient-separated based on their hydrophobic/hydrophilic interactions. The RPC column (3) may include a hydrophobic or non-polar stationary phase, such as octadecyl silane (C18). The above-described Solvent A from Pump A and/or Solvent B form Pump B may serve as a gradient mobile phase. An exemplary Solvent A includes 2 vol. % acetonitrile, 98 vol. % water and 0.1 vol. % formic acid, and an exemplary Solvent B includes 90 vol. % acetonitrile, 10 vol. % water and 0.1 vol. % formic acid, such that Solvent A is more hydrophilic than Solvent B. Solvent A and Solvent B may be combined in different relative amounts. For example, a combined solvent may be formed with 2 vol. % Solvent A and 98 vol. % Solvent B, or with 98 vol. % Solvent A and 2 vol. % Solvent B. The gradient may be achieved over time by increasing the concentration of one solvent (e.g., Solvent B) and decreasing the concentration of the other solvent (e.g., Solvent A). More hydrophilic or polar materials will elute from the RPC column (3) first with Solvent A, while more hydrophobic or non-polar materials will be retained within the RPC column (3) and elute later with Solvent B.

The peptide fragments from the RPC column (3) may then be directed to a mass spectrometer (MS) for peptide sequence analysis. The peptide sequence may be compared with sequences in DNA and protein databases to find matching signature sequences that can be used to identify the protein parent from which the peptide sequence was derived, for example. With Valve 1 in the second position (FIG. 2B), the material from the RPC column (3) illustratively enters Port 6, crosses over to Port 5, and continues to the mass spectrometer (MS). Alternatively, with Valve 1 in the first position (FIG. 2A), the material from the RPC column (3) illustratively enters Port 6, crosses over to Port 7, and is then diverted to waste.

It is also within the scope of the present disclosure that the system may include a UV absorbance monitor for further analysis.

### II. Digestion Buffer

An enhanced digestion buffer solution is provided for use with an immobilized enzyme reactor, such as an immobilized trypsin reactor. In addition to controlling the pH in the immobilized enzyme reactor, such as between pH 7 and pH 9, the digestion buffer solution may cause a significant change in the reaction environment of the immobilized enzymes to increase recovery and reduce carry-over of peptide fragments from the immobilized enzymes. Also, the digestion buffer solution may avoid impeding retention of peptide fragments during subsequent desalting and separation processes. Thus, the digestion buffer solution may facilitate the automated, reproducible, fast, multistep flow through the digestion column (1) and the subsequent desalting column (2) and RPC column (3) of Section I above, for example.

According to an exemplary embodiment of the present disclosure, the enhanced digestion buffer solution includes at least one component of high buffering capacity, such as between pH 7 and pH 9. Within the immobilized digestion column (1), for example, the component having the high buffering capacity may mitigate non-specific hydrophobic and/or electrostatic interactions between the digestion products and the digestion column (1) (e.g., the immobilized enzymes, the solid-support material) to increase recovery of the digestion products from the digestion column (1). Exemplary components having a high buffering capacity that may be suitable for use in the digestion buffer solution include, for example, tris(hydroxymethyl)methylamine (Tris), phosphate buffer saline (PBS), 3-{[tris(hydroxymethyl)methyl]amino}propanesulfonic acid (TAPS), N-tris(hydroxymethyl)methylglycine (Tricine), 3-[N-Tris(hydroxymethyl)methylamino]-2-hydroxypropanesulfonic Acid (TAPSO), 4-2-hydroxyethyl-1-piperazineethanesulfonic acid (HEPES), 2-{[tris(hydroxymethyl)methyl]amino}ethanesulfonic acid (TES) 3-(N-morpholino)propanesulfonic acid (MOPS), piperazine-N,N′-bis(2-ethanesulfonic acid) (PIPES), dimethylarsinic acid (Cacodylate), saline sodium citrate (SSC), 3-(Cyclohexylamino)-1-propanesulfonic acid (CAPS), 2-(Cyclohexylamino)ethanesulfonic acid (CHES), 3-[4-(2-hydroxyethyl)piperazin-1-yl]propane-1-sulfonic acid (HEPPS), 2-(N-morpholino)ethanesulfonic acid (MES), 1,3-bis(tris(hydroxymethyl)methylamino)propane (Bis-Tris), 2,2-bis(hydroxymethyl)-2,2′,2″-nitrilotriethanol (also Bis-Tris), and the like. Tris may be provided as a Tris Base and/or as a Tris Acid, such as Tris hydrochloride (Tris HCl). Tris Base may have a pKa of 8.1 at 25° C. Tris Base and Tris Acid may be provided in combination to ensure an adequate buffering capacity.

According to another exemplary embodiment of the present disclosure, the enhanced digestion buffer solution includes at least one component of high ionic strength. Within the immobilized digestion column (1), for example, the component having the high ionic strength may mitigate non-specific hydrophobic and/or electrostatic interactions between the digestion products and the digestion column (1) (e.g., the immobilized enzymes, the solid-support material) to increase recovery of the digestion products from the digestion column (1). Exemplary components having a high ionic strength that may be suitable for use in the digestion buffer solution include, for example, sodium chloride, magnesium chloride, magnesium sulfate, and the like.

Components of high buffering capacity and/or high ionic strength should be present in the digestion buffer solution at a concentration that is sufficient to improve recovery from the immobilized digestion column (1), for example. This concentration may be determined for a single component or a plurality of components in combination. At relatively low concentrations, such as 0.05 M (50 mM) and 0.1 M (100 mM), the components have not been shown to adequately improve recovery from the immobilized digestion column (1). Thus, it is believed that the components should be present in the digestion buffer solution at relatively high concentrations greater than 0.05 M (50 mM) and 0.1 M (100 mM), for example. In certain embodiments, the components may be present in the digestion buffer solution at a concentration of 1 M or more. At relatively high concentrations, the digestion buffer solution has been shown to improve recovery from the immobilized digestion column (1), which may be due to the fact that the high organic content of the digestion buffer solution is negating or mitigating hydrophobic and/or electrostatic interactions in the digestion column (1), as discussed above. In other embodiments, the components may be present in the digestion buffer solution at concentrations less than 1 M, such as concentrations as low as 0.9 M, 0.8 M, 0.7 M, 0.6 M, 0.5 M, or less, for example.

An exemplary digestion buffer solution includes at least about 1 M Tris, which was shown to substantially increase recoveries of digested peptide products from immobilized enzyme reactors when compared to standard 0.05 M (50 mM) Tris buffers. The digestion buffer solution may also include one or more ingredients to mitigate ionic interactions, such as NaCl, one or more ingredients to enhance trypsin activity, such as CaCl₂, and one or more ingredients to mitigate hydrophobic interactions, such as isopropyl alcohol (IPA). For example, the digestion buffer solution may include: 127 g Tris HCl (0.81 M), 68 g Tris Base (0.56 M) (for a combined total of 1.37 M Tris), 8.77 g NaCl (0.15 M), 1.1 g CaCl₂ (0.01 M), and 20 mL IPA (2 vol. %) present in enough water to bring the total volume of the digestion buffer solution to 1 L. In this embodiment, Tris is the majority solute or additive in the IPA and water solvent, both in terms of weight and molar concentration.

### III. Miniaturized Digestion Columns

The digestion column (1) described in Section I above may be miniaturized to reduce the total surface area of the immobilized enzyme contained therein, which may minimize non-specific interactions in the digestion column (1) and optimize digestion yields from the digestion column (1). Additionally, miniaturizing the digestion column (1) may shorten the digestion cycle, shorten the washing cycle, reduce manufacturing costs, and reduce other ingredient costs (e.g., the digestion buffer). Reducing the internal diameter of the digestion column (1) from 4.6 mm to 2.1 mm or less, for example, may shorten the process and reduce costs without impacting carry-over. One may wish to apply the technology described herein on devices having a size which ranges from the conventional lab reactor down to micro-sized reactors, such as microfluidic devices.

### IV. Parallel Processing

The techniques described herein may reduce carry-over in the digestion column (1), thereby reducing the amount of washing required between samples and reducing time spent in the digestion column (1). In view of these process improvements, two or more samples may be simultaneously processed in the system described in Section I above. For example, while one sample is undergoing separation in the RPC column (3), another sample may be undergoing digestion in the digestion column (1). The ability to process two or more samples at the same time may substantially reduce the total time required to process a large batch of samples.

Such simultaneous or parallel processing may be facilitated by providing multiple desalting columns (2a) and (2b). While material is entering the second desalting column (2b) to undergo desalting, material that has already undergone desalting may be exiting the other desalting column (2a). For example, with Valve 2 in the first position (FIG. 2A), material from the digestion column (1) may be entering Port 1, crossing over to Port 10, and entering the second desalting column (2b). At the same time, material may be exiting the first desalting column (2a), entering Port 5, crossing over to Port 4, traveling to Port 9, crossing over to Port 8, and continuing to the RPC column (3).

### V. Direct Digestion of Unfractionated Biomatrices

The techniques described herein may improve recovery from the digestion column (1), thereby allowing for direct digestion of proteins from complex biomatrices in the digestion column (1) without pre-fractionation. Suitable unfractionated biomatrices may include, for example, blood serum, blood plasma, urine, cerebral spinal fluid, and the like. In cases where the concentration of the target protein in the sample is sufficiently high, an unfractionated sample may be diluted, directly digested in the digestion column (1), and then analyzed using MS. By avoiding pre-fractionation (e.g., affinity selection) to remove debris and other materials from the sample before the digestion column (1), the process may be simplified and shortened. Digestion in the digestion column (1) may occur in less than 10 minutes, 8 minutes, 6 minutes, or 4 minutes, and the overall processing time may occur in less than 24 minutes, 22 minutes, 20 minutes, or 18 minutes, for example.

### VI. Software

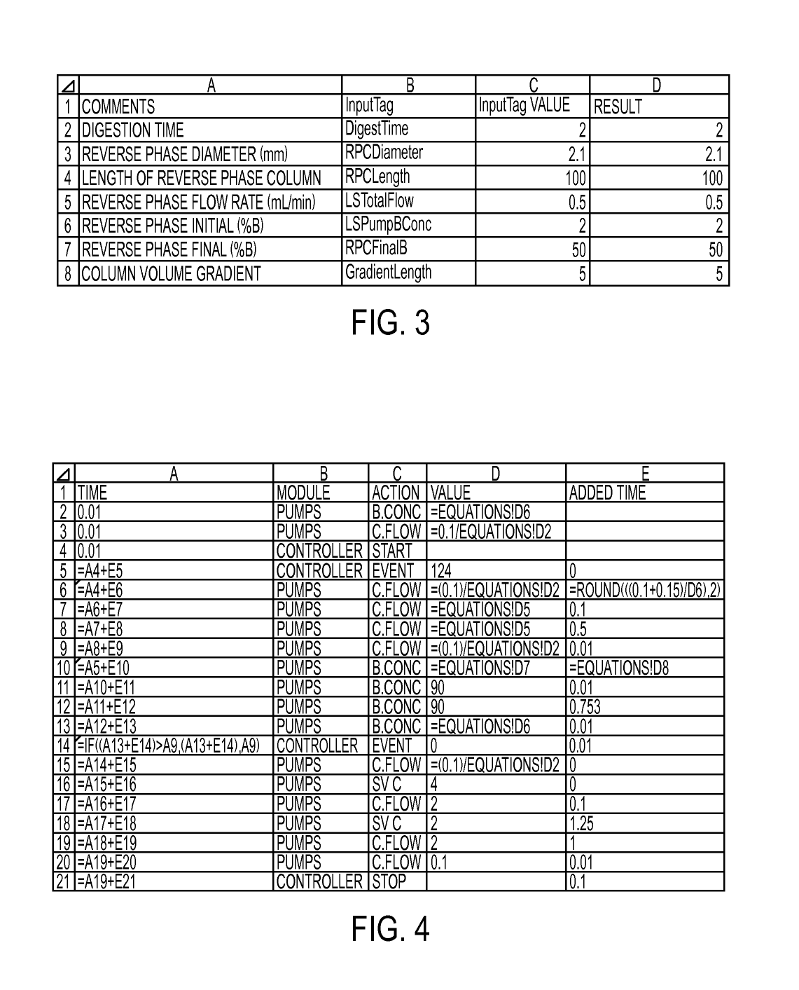

The system described in Section I may be operated by a controller, which may be in the form of a suitably programmed microprocessor or computer. The controller may be programmed to accept a variety of user inputs, such as processing times, oven temperatures, and other conditions. In the illustrated embodiment of FIG. 3, for example, the controller accepts the following user inputs: digestion time (in minutes), the diameter of the RPC column (in mm), the length of the RPC column (in mm), the flow rate through the RPC column (in mL/min), the initial concentration of the Solvent B from Pump B (%), the final concentration of the Solvent B from Pump B (%), and the gradient length in the RPC column (in minutes). The controller illustratively stores the user inputs in a database entitled “Equations.”

The controller then performs a set of calculations using the user inputs from FIG. 3 to produce a time program, as shown in FIG. 4. The controller follows the time program to automatically perform digestion, desalting, and reverse phase separation. With respect to FIG. 1, for example, the controller may follow the time program to control operation of the auto-sampler syringe, Pumps A, B, and C, Valves 1 and 2, and/or the mass spectrometer.

At a start time (0.01 s), Pump C is set to deliver its digestion solution a flow rate that will achieve the user's desired digestion time by dividing the internal volume of the digestion column (1) by the user's desired digestion time (See Cell D3 in FIG. 4). In this particular example, the digestion column (1) has an internal volume of 0.1 mL (i.e., 2.1 mm internal diameter and 30 mm length). To account for dead volume in the system, the Pump C will run longer than the user's desired digestion time. The time is calculated by dividing the combined volume of the digestion column (1) and the dead volume by the previously calculated flow rate (See Cell E6 in FIG. 4). In this particular example, the dead volume in the system is 0.15 mL.

In order to maintain compatibility with a range of flow rates in the RPC column (3), Pump C may be switched to deliver its solution at the same flow rate as the RPC column (3) (See Cells D7 and D8 in FIG. 4). Following this step, the flow rate through the digestion column (1) may be returned to the calculated flow rate described above (See Cell D9 in FIG. 4), where it is maintained through the reverse phase gradient (See Cell D15 in FIG. 4).

Also at the start time, Pump B is set to deliver the Solvent B at the user's desired initial concentration (See Cell D2 in FIG. 4) for desalting. After the user's desired gradient time, the Pump B will shift to deliver user's desired final concentration (See Cells D10 and E10 in FIG. 4) for reverse phase separation. After the reverse phase gradient is performed in the RPC column (3), Pump B will return to delivering the Solvent B at the user's desired initial concentration (See Cell D13 in FIG. 4).

The controller may also perform appropriate calculations to delay the start of a subsequent step until completion of the prior steps in the proper order.

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

### Examples

The following examples are meant to illustrate, but in no way to limit, the claimed invention.

### 1. Example 1

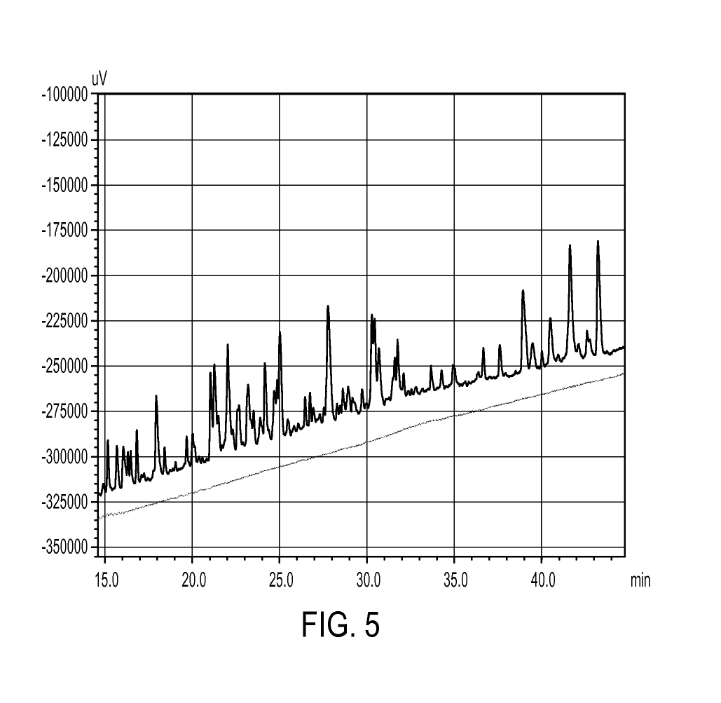

A first experiment was performed to evaluate recovery for a particular digestion buffer containing 127 g Tris HCl (0.81 M), 68 g Tris Base (0.56 M) (for a combined total of 1.37 M Tris), 8.77 g NaCl (0.15 M), 1.1 g CaCl₂ (0.01 M), and 20 mL IPA (2 vol. %) present in enough water to bring the total volume of the digestion buffer solution to 1 L. 20 μg of Transferrin was digested in an immobilized enzyme column for 4 minutes with the digestion buffer. After digestion, the material was desalted with 2 vol. % of a Solvent B (containing 90 vol. % acetonitrile, 10 vol. % water, and 0.1 vol. % formic acid) and 98 vol. % of a Solvent A (containing 2 vol. % acetonitrile, 98 vol. % water, and 0.1 vol. % formic acid) for approximately 1 minute, and then the material was subjected to reverse phase separation with 2-50 vol. % Solvent B for 40 minutes. A UV trace of the resulting reverse phase chromatogram (monitoring absorbance at 214 nM) is shown in FIG. 5. In a subsequent blank run, carry-over was absent, which indicates high recovery from the immobilized enzyme column.

### 2. Example 2

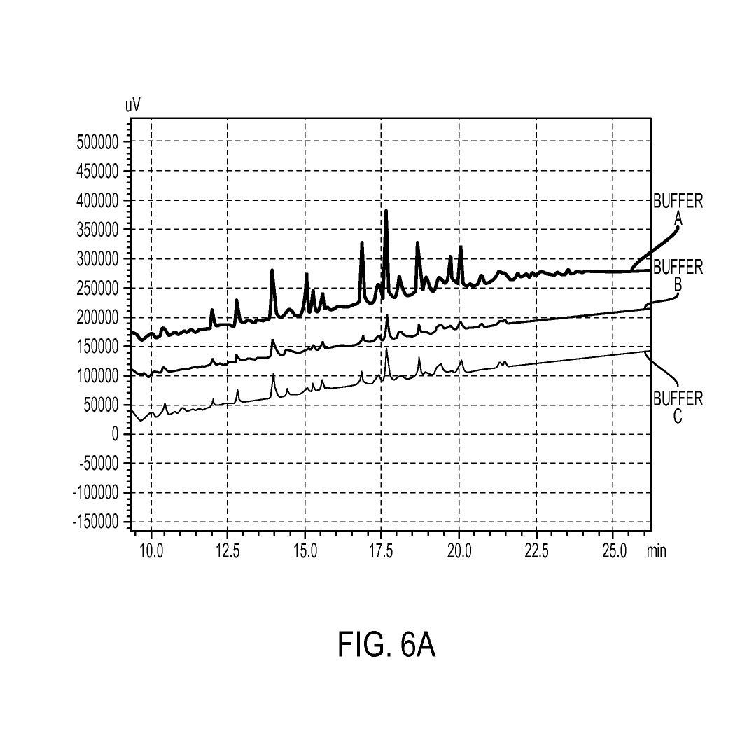

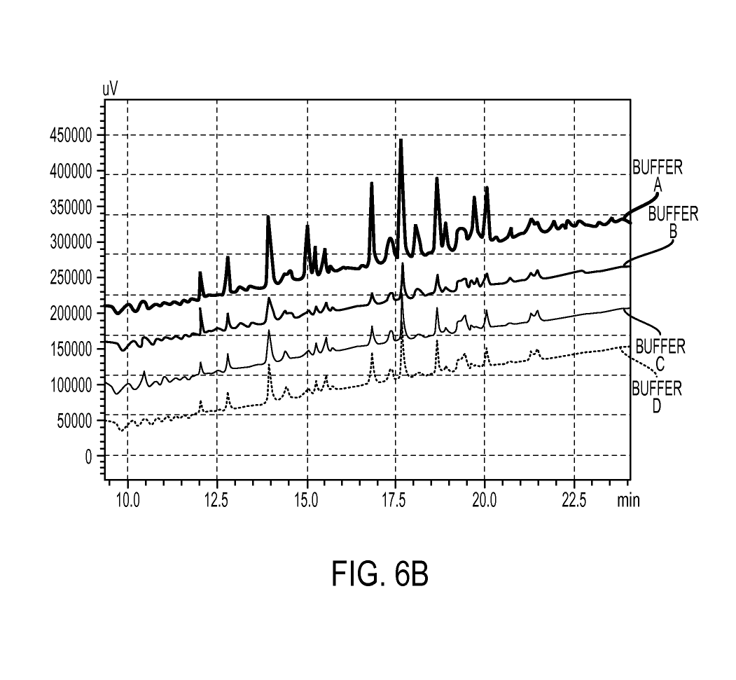

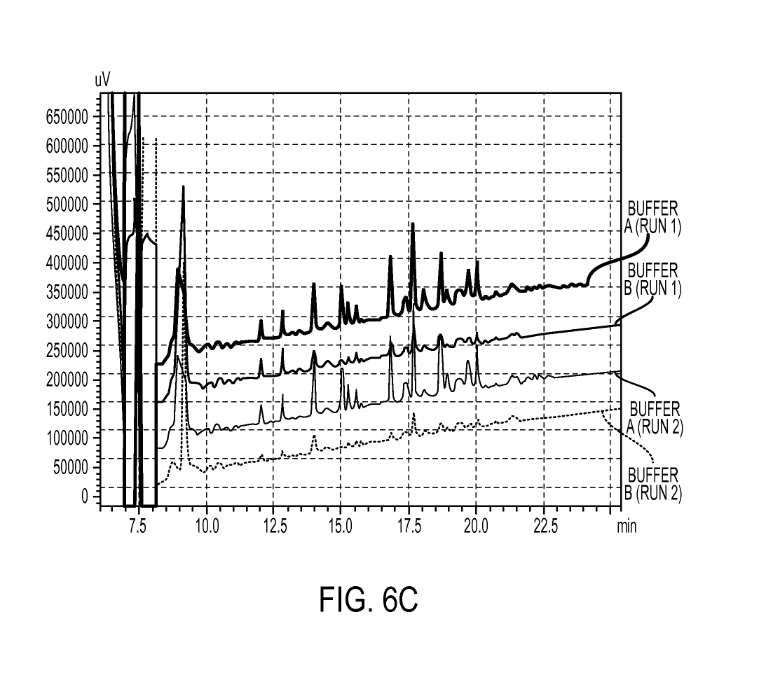

Another experiment was performed to evaluate recoveries for various digestion buffers. For each digestion buffer, 25 μg of hemoglobin variant S was digested in an immobilized enzyme column for 4 minutes. After digestion, the material was desalted with 2 vol. % of the same Solvent B from Example 1 and 98 vol. % of the same Solvent A from Example 1 for approximately 1 minute, and then the material was subjected to reverse phase separation with 2-50 vol. % Solvent B for 20 minutes. A UV trace of each resulting reverse phase chromatogram (monitoring absorbance at 214 nM) is shown in FIGS. 6A-6C.

A first set of digestion buffers evaluated in this experiment included:

Buffer A=1 M Tris, 150 mM NaCl, 10 mM CaCl₂, 2 vol. % IPA

Buffer B=50 mM Tris

Buffer C=50 mM Tris, 150 mM NaCl

The results are presented in FIG. 6A. Based on the relative size of the peaks in FIG. 6A, Buffer A achieved the highest recovery, followed by Buffer C, with Buffer B exhibiting the lowest recovery and the most carry-over. Therefore, the presence of 1 M Tris in Buffer A is believed to improve recovery over 50 mM Tris in Buffers B and C. The presence of 150 mM NaCl in Buffers A and C may also improve recovery over Buffer B, but to a lesser extent than 1 M Tris.

A second set of digestion buffers evaluated in this experiment included:

Buffer A=1 M Tris, 150 mM NaCl, 10 mM CaCl₂, 2 vol. % IPA

Buffer B=50 mM Tris

Buffer C=50 mM Tris, 150 mM NaCl

Buffer D=50 mM Tris, 150 mM NaCl, 2 vol. % IPA

The results are presented in FIG. 6B. Based on the relative size of the peaks in FIG. 6B, Buffer A again achieved the highest recovery. The addition of 2 vol. % IPA to Buffer D did not significantly improve recovery compared to Buffer C.

A third set of digestion buffers evaluated in this experiment included:

Buffer A (Run 1)=1 M Tris, 150 mM NaCl, 10 mM CaCl₂, 2 vol. % IPA

Buffer B (Run 1)=50 mM Tris

Buffer A (Run 2)=1 M Tris, 150 mM NaCl, 10 mM CaCl₂, 2 vol. % IPA

Buffer B (Run 2)=50 mM Tris

The results are presented in FIG. 6C. Based on the relative size of the peaks in FIG. 6C, Buffer A again achieved the highest recovery in both Runs 1 and 2.

### 3. Example 3

Another experiment was performed to evaluate recovery for the same digestion buffer as Example 1. Samples were prepared by diluting Cytochrome C at various concentrations with 10 vol. % Blocker Casein, as set forth in Table 1 below. Each sample was digested in an immobilized enzyme column for 2 minutes with the digestion buffer. After digestion, the material was desalted with 2 vol. % of the same Solvent B from Example 1 and 98 vol. % of the same Solvent A from Example 1 for approximately 1 minute, and then the material was subjected to reverse phase separation with 10-70 vol. % Solvent B for 5 minutes. Each sample was then subjected to selective reaction monitoring (SRM) of the peptide MIFAGIK precursor mass m/z 779.54 and fragment ions 520.2 (b5), 535.3 (y5) and 633.4 (b6). Each protein sample injection was followed by a blank injection of 10 vol. % Blocker Casein. Carry-over was determined by dividing the peak area of the post-run blank by the peak area of the protein sample run. The low carry-over results presented in Table 1 below indicate high recovery of the digested material from the immobilized enzyme column.

TABLE 1

| Protein Samples | Monitoring of Peptide MIFAGIK | | | |
|---|---|---|---|---|
| Concentration | Pre-Blank | Sample | Post-Run Blank | Carry-Over |
| (μg/mL) | Area | Area | Area | (%) |
| 1 | 0 | 5,466 | 0 | 0 |
| 10 | 0 | 152,094 | 133 | 0.09 |
| 100 | 0 | 703,172 | 641 | 0.09 |

### 4. Example 4

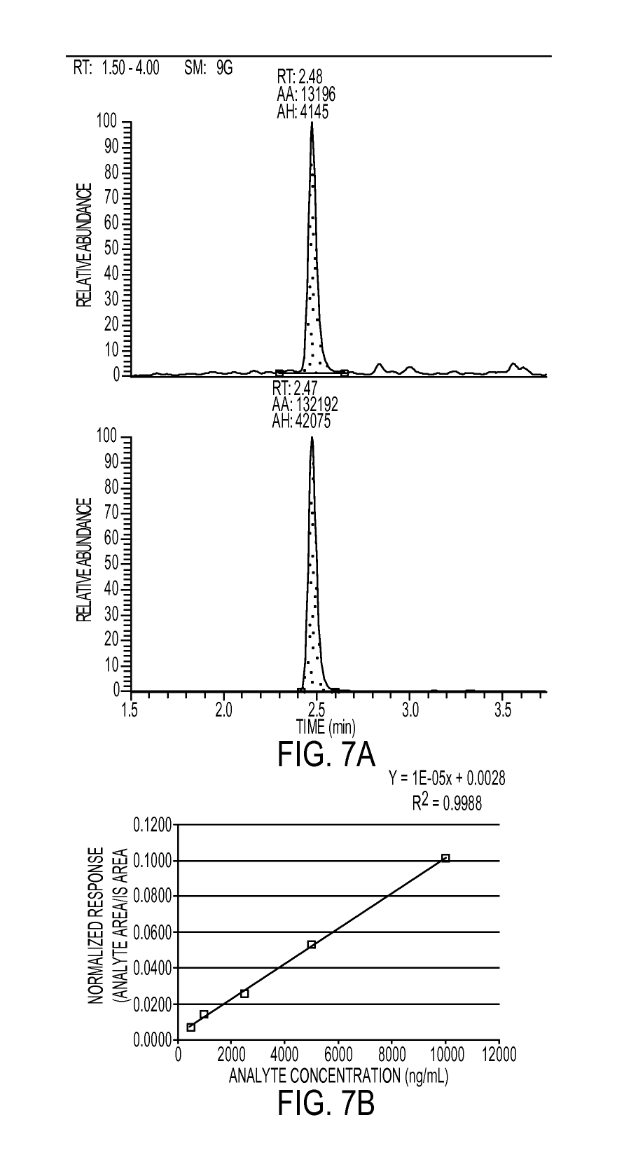

Another experiment was performed to evaluate recovery for the same digestion buffer as Example 1. Plasma samples were spiked with insulin at various concentrations ranging from 500 to 10,000 ng/mL, as set forth in Table 2 below. Following a 10-fold dilution with 2M urea, each sample was injected directly into an immobilized enzyme column and was digested for 4 minutes at 50° C. with the digestion buffer. After digestion, each sample was desalted with 2 vol. % of the same Solvent B from Example 1 and 98 vol. % of the same Solvent A from Example 1 for approximately 1 minute, and then each sample was subjected to reverse phase separation in a C18 column with 10-70 vol. % Solvent B for 5 minutes. From the reverse phase column, the samples were directed to an ion-trap mass spectrometer using positive electrospray ionization. A labeled synthetic peptide that mimicked the c-terminal sequence of insulin was used as an internal standard (IS) to normalize for variability in mass spectrometric ionization. Quantification of insulin was conducted by selective reaction monitoring (SRM) of the transitions of m/z 859.4→(841.5+616.3) for insulin and m/z 869.4→(851.5+626.3) for the IS, as shown in FIG. 7A. The insulin response was divided by the internal standard response to give a “normalized” result.

TABLE 2

| | Normalized Standard Curve (Analyte Area/IS Area) | | | | |
|---|---|---|---|---|---|
| Concentration (ng/mL) | 500 | 1,000 | 2,500 | 5,000 | 10,000 |
| Run 1 | 0.0067 | 0.0155 | 0.0290 | 0.0549 | 0.1029 |
| Run 2 | 0.0062 | 0.0133 | 0.0254 | 0.0539 | 0.1046 |
| Run 3 | 0.0077 | 0.0161 | 0.0266 | 0.0549 | 0.0999 |
| Run 4 | 0.0082 | 0.0141 | 0.0256 | 0.0508 | 0.1018 |
| Run 5 | 0.0059 | 0.0154 | 0.0253 | 0.0541 | 0.1014 |
| Run 6 | 0.0073 | 0.0144 | 0.0273 | 0.0550 | 0.1053 |
| Run 7 | 0.0073 | 0.0144 | 0.0244 | 0.0535 | 0.1016 |
| Run 8 | 0.0072 | 0.0124 | 0.0237 | 0.0499 | 0.0970 |
| Average | 0.0071 | 0.0144 | 0.0259 | 0.0534 | 0.1018 |
| StDev | 0.0008 | 0.0012 | 0.0017 | 0.0019 | 0.0026 |
| CV (%) | 10.66 | 8.34 | 6.50 | 3.65 | 2.58 |

As shown in FIG. 7B, the assay exhibited linearity from 500 and 10,000 ng/mL, which demonstrates exceptional reproducibility and reliability and dramatically reduced sample processing time from 24 hours to 18 minutes

### 5. Example 5

Another experiment was performed to evaluate recovery from a miniaturized immobilized trypsin column having an internal diameter of 2.1 mm compared to an internal diameter of 4.6 mm. Samples were prepared from 1 μg/mL insulin stock in 10 vol. % Blocker Casein. Each sample was digested in an immobilized enzyme column for 4 minutes. After digestion, the material was desalted with 2 vol. % of the same Solvent B from Example 1 and 98 vol. % of the same Solvent A from Example 1 for approximately 1 minute, and then the material was subjected to reverse phase separation with 10-70 vol. % Solvent B for 5 minutes. Each sample was then subjected to selective reaction monitoring (SRM) of the peptide GFFYTPK monitoring m/z 859.4→(616.3+841.4) and m/z 869.5→(626.4+851.5) for the isotope-labeled internal standard (IS). Each protein sample injection was followed by a blank injection of 10 vol. % Blocker Casein. Carry-over was determined by dividing the peak area of the post-run blank by the peak area of the protein sample run. The carry-over results are presented in Table 3 below, showing substantially less carry-over in the 2.1 mm column than in the 4.6 mm column.

TABLE 3
4.6 mm Internal Diameter

| | |
|---|---|
| Pre-Run Peak Area | 83.76 |
| Protein Sample Peak Area | 38,372 |
| Post-Run Peak Area | 200 |
| Carry-Over (%) | 0.52 |

2.1 mm Internal Diameter

| | |
|---|---|
| Pre-Run Peak Area | 0 |
| Protein Sample Peak Area | 117,564 |
| Post-Run Peak Area | 91.29 |
| Carry-Over (%) | 0.08 |

What is claimed is:

1. An immobilized enzyme reactor comprising:
a protein sample; and
a digestion buffer solution comprising:
at least one buffer component having a high buffering capacity, the at least one buffer component present in the digestion buffer solution at a concentration greater than 0.1 M.

2. The immobilized enzyme reactor of claim 1, wherein the at least one buffer component is present in the digestion buffer solution at a concentration of at least about 1 M.

3. The immobilized enzyme reactor of claim 1, wherein the at least one buffer component comprises Tris.

4. The immobilized enzyme reactor of claim 1, wherein the at least one buffer component comprises Tris Acid and Tris Base.

5. The immobilized enzyme reactor of claim 4, wherein the digestion buffer solution comprises:
0.81 M Tris HCl, and
0.56 M Tris Base.

6. The immobilized enzyme reactor of claim 1, wherein the digestion buffer solution further comprises:
NaCl,
CaCl₂, and
a solvent comprising water and isopropyl alcohol.

7. An immobilized enzyme reactor comprising:
a protein sample; and
a digestion buffer solution comprising:
a solvent; and
at least one buffer component having a high buffering capacity, the at least one buffer component being present as a majority solute in the solvent.

8. The immobilized enzyme reactor of claim 7, wherein the digestion buffer solution comprises NaCl and CaCl₂ present as minority solutes in the solvent.

9. The immobilized enzyme reactor of claim 7, wherein the digestion buffer solution consists essentially of the solvent, Tris, NaCl, and CaCl₂.

10. The immobilized enzyme reactor of claim 7, wherein the solvent of the digestion buffer solution comprises water and isopropyl alcohol.

11. The immobilized enzyme reactor of claim 7, wherein the immobilized enzyme reactor comprises an immobilized trypsin reactor.

12. The immobilized enzyme reactor of claim 7, wherein the at least one buffer component comprises Tris Acid and Tris Base.

13. The immobilized enzyme reactor of claim 12, wherein the digestion buffer solution comprises the solvent and:
0.81 M Tris HCl,
0.56 M Tris Base,
0.15 M NaCl, and
0.01 M CaCl₂.

14. A method for analyzing a protein comprising the steps of:
preparing a sample from an unfractionated biomatrix comprising the protein;
transporting the sample directly to an immobilized enzyme reactor to digest the protein into peptides without first fractionating the unfractionated biomatrix;
automatically transporting the peptides from the immobilized enzyme reactor to a desalting apparatus;
automatically transporting the peptides from the desalting apparatus to a reverse phase separation apparatus; and
analyzing the peptides using a mass spectrometer.

15. The method of claim 14, wherein the unfractionated biomatrix comprises one of blood serum, blood plasma, urine, and cerebral spinal fluid.

16. The method of claim 14, wherein the sample travels through the immobilized enzyme reactor, the desalting apparatus, and the reverse phase separation apparatus in less than about 24 minutes.

17. A system for analyzing a protein comprising:
an immobilized enzyme reactor;
a first desalting apparatus in communication with the immobilized enzyme reactor;
a second desalting apparatus in communication with the immobilized enzyme reactor and arranged in parallel with the first desalting apparatus;
a reverse phase separation apparatus in communication with the first and second desalting apparatuses; and
a mass spectrometer in communication with the reverse phase separation apparatus.

18. The system of claim 17, wherein the first desalting apparatus is configured to receive a first sample from the immobilized enzyme reactor while the second desalting apparatus delivers a second sample to the reverse phase separation apparatus.

19. The system of claim 17, wherein the immobilized enzyme reactor comprises an immobilized trypsin reactor.

20. The system of claim 17, further comprising a valve in communication with the first desalting apparatus and the second desalting apparatus, the valve having a first position that directs the sample from the immobilized enzyme reactor to the first desalting apparatus and a second position that directs the sample from the immobilized enzyme reactor to the second desalting apparatus.
